(19) United States
(12) Patent Application Publication
(10) Pub. No.: US 2006/0149102 A1
(43) Pub. Date: Jul. 6, 2006

(54) Oxychlorination of olefins and aromatics by a novel concept of fluidized bed reaction

(75) Inventors: Andreas Voight, Wien (AT); Franz Winter, Baden (AT); Martin Kozek, Gaaden (AT)

Correspondence Address: EDWARDS & ANGELL, LLP, P.O. BOX 55874, BOSTON, MA, 02205, US

(73) Assignee: Austria Wirtschaftsservice Gesellschaft mgH, Vienna (AT)

(21) Appl. No.: 10/525,419

(22) PCT Filed: Aug. 8, 2003
(86) PCT No.: PCT/EP03/08846

§ 371 Date: Sep. 9, 2005

(30) Foreign Application Priority Data
Aug. 23, 2002 (DE) 102 38 811.3

(51) Int. Cl. C07C 17/15; C07C 17/152

(52) U.S. Cl. 570/243

(57) ABSTRACT

A continuous process to oxychlorinate olefins and aromatics is described, comprising the conversion of olefins and aromatics as component (a) with oxygen and hydrogen chloride as component (b) in the presence of a solid cuprous/cupric salt catalyst in a reactor, characterized in that components (a) and (b) are fed separately from each other in spatial terms into reaction zones and regeneration zones of the reactor, where the reaction zone shows a higher concentration of the catalyst in its oxidized form at the solids entry point than at the solids exit point, and the regeneration zone shows a higher concentration of the catalyst in its reduced form at the solids entry point than at its solids exit point, and where component (a) is fed into the reaction zones and component (b) is fed into the regeneration zones.

The present invention concerns a novel process to oxychlorinate olefins and aromatics by using a special fluidized bed reactor concept.

Oxychlorinating olefins and aromatics by way of oxygen and hydrogen chloride is a known process, described, i.a., in Ullmann's Encyclopaedia of Industrial Chemistry, Wiley-VCH Verlag GmbH, Germany, 2002, chapter 2.3; and S. Sai Prasad, B. S. Pradad, M. S: Ananth, Parameter Estimation in Fixed-Bed Reactor Operating under Unsteady Stat: Oxychlorination of Ethylene, Ind. Eng. Chem. Res., vol. 40, pp. 5487-5495, Indian Institute of Chemical Technology, 2001; U.S. Pat. No. 3,148,222; and in Beyer, Walter, Lehrbuch der Organischen Chemie, S. Hirzel Verlag, Stuttgart.

These processes are performed using heterogeneous catalysis with a cuprous/cupric salt catalyst (cf., e.g., S. Wachi, Yousuke Asai, Kinetics of 1,2-Dichlorethane Formation from Ethylene at Cupric Chloride, Ind. Eng. Chem. Res., vol. 33, pp. 259-264, Japan, 1994).

Oxychlorination of ethylene in particular is of industry-scale importance. This is the subject of DE 43 03 086 and JP 59016835. It yields 1,2-dichloroethane (ethylene dichloride, EDC) by using a cupric chloride catalyst, at the following overall reaction:
C₂H₄+2 HCl+½ O₂→C₂H₄Cl₂+H₂O

In fixed and fluidized bed reactors, conversion is typically at 200° C. to 240° C. and increased pressure. The synthesis is in the form of a heterogeneous catalyst reaction, with CuCl₂ as the catalyst. This CuCl₂ is applied to a support (frequently Al₂O₃) as a percentage by mass of 3-7%. The educts ethylene, oxygen (in the form of air or pure oxygen) and hydrogen chloride are jointly fed to the lowest level of the reactor. In order to obtain information on the conversion of the educts, the system is adjusted to produce a small stoichiometric excess of ethylene and oxygen. The overhead product of the oxychlorination reactor is 1,2-dichloroethane and steam as the main components and non-converted ethylene, oxygen and HCl. When directly cooled with water in the downstream quenching unit, hydrogen chloride is washed out from the mix. Once the product and water have condensed, the product may be extracted. Non-condensable gases are either recycled as circulating gas or extracted as waste gas. Part of the circulating gas always needs to be extracted so that the system pressure can be maintained. The product still contains dissolved water which is extracted by distillation. In the process known to the state of the art, the reactor is operated either by the so-called “circulating gas mode” using pure oxygen or the so-called “air mode” which uses air as a source of oxygen.

Both processes yield oxidation by-products such as CO₂ and CO, which reduce the raw material yield, generate costs of disposal and, being waste gas, are a burden on the environment. The catalyst is not used to its optimum extent.

The present invention is based on the objective of providing a process to oxychlorinate olefins and aromatics whereby the quantity of by-products produced by the reaction is reduced, the loss of olefins/aromatics and oxygen as well as the volume of waste gas is minimized and the purity of the product generated is increased so that the cost of cleaning the product (e.g. by distillation) is reduced.

The subject matter of the invention is a continuous process to oxychlorinate olefins and aromatics, comprising the conversion of olefins and aromatics as component (a) with oxygen and hydrogen chloride as component (b) in the presence of a solid cuprous/cupric salt catalyst in a reactor, characterized in that components (a) and (b) are fed separately from each other in spatial terms into reaction zones and regeneration zones of the reactor, where the reaction zone shows a higher concentration of the catalyst in its oxidized form at the solids entry point than at the solids exit point, and the regeneration zone shows a higher concentration of the catalyst in its reduced form at the solids entry point than at its solids exit point, and where component (a) is fed into the reaction zones and component (b) is fed into the regeneration zones.

The invention is explained in more detail in the enclosed figures:

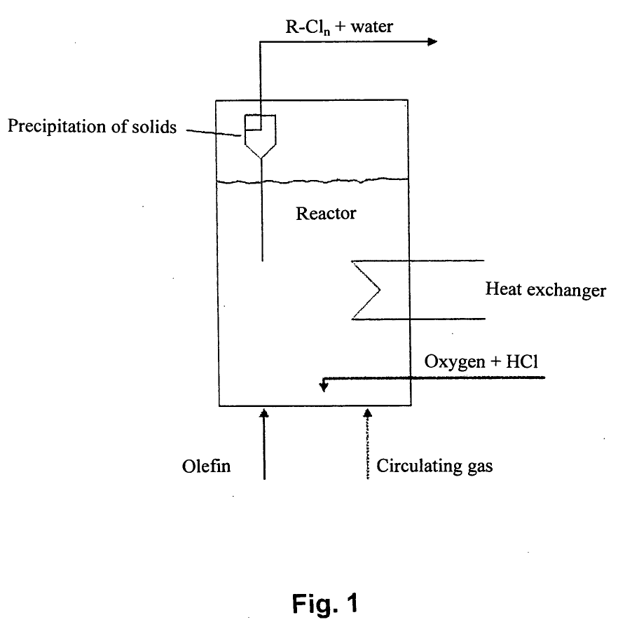

FIG. 1 illustrates the above circulating gas mode used for state-of-the-art oxychlorination.

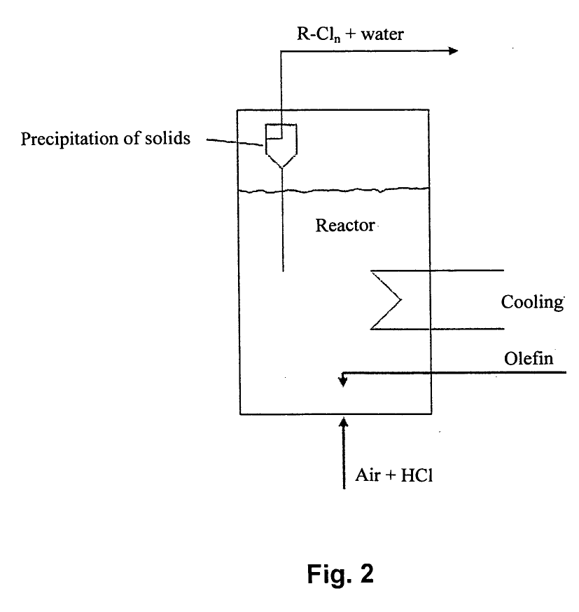

FIG. 2 illustrates the air mode.

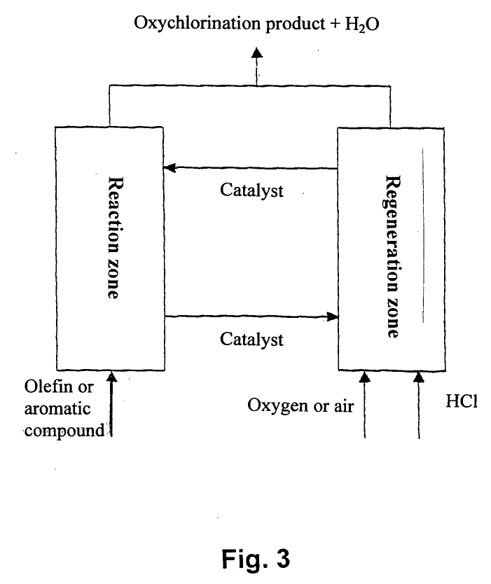

FIG. 3 shows a schematic diagram of the reactor arrangement to carry out the process according to the invention, where the reactor comprises so-called reaction zones and regeneration zones.

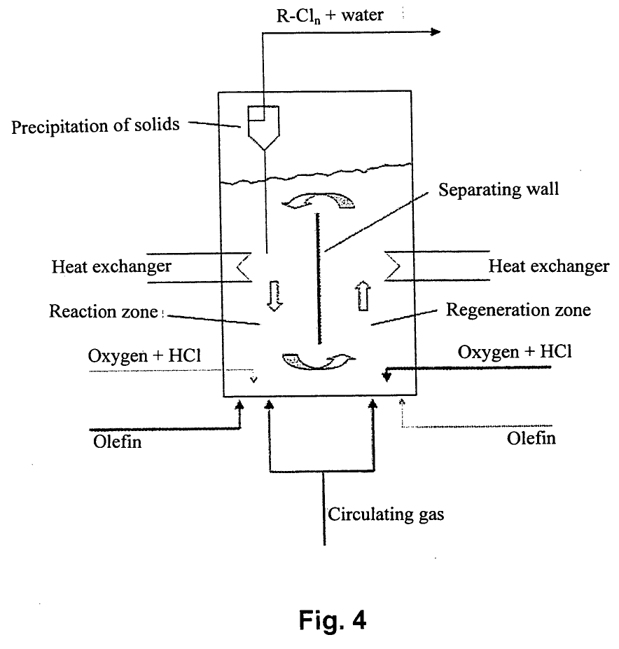

FIG. 4 shows an embodiment of the reactor to carry out the process according to the invention using internal catalyst circulation.

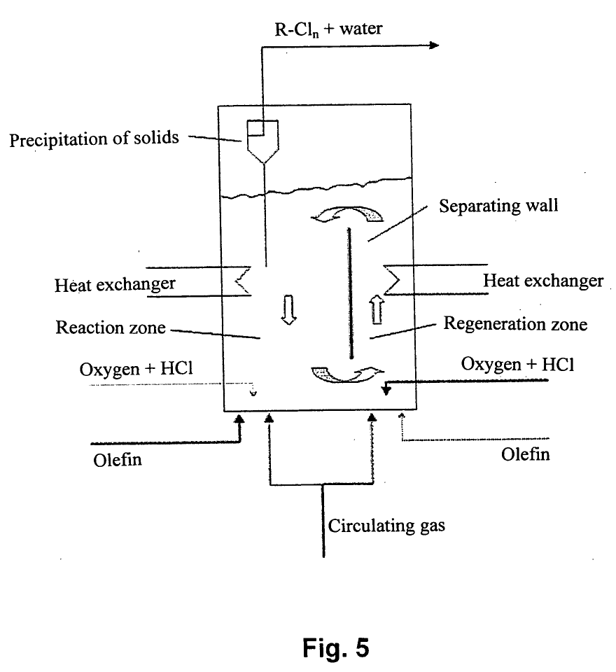

FIG. 5 shows another embodiment of the reactor to carry out the process according to the invention using internal catalyst circulation.

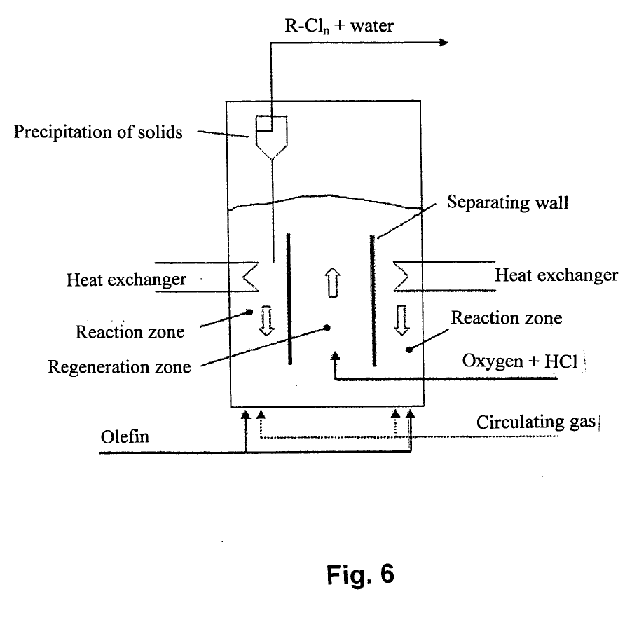

FIG. 6 shows another embodiment of the reactor to carry out the process according to the invention using internal catalyst circulation.

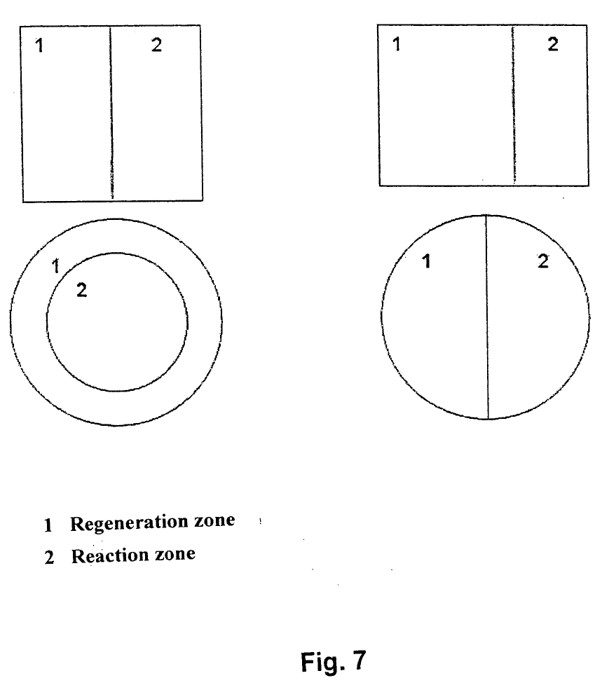

FIG. 7 shows cross-sectional shapes for the reactor pursuant to FIGS. 4, 5 and 6.

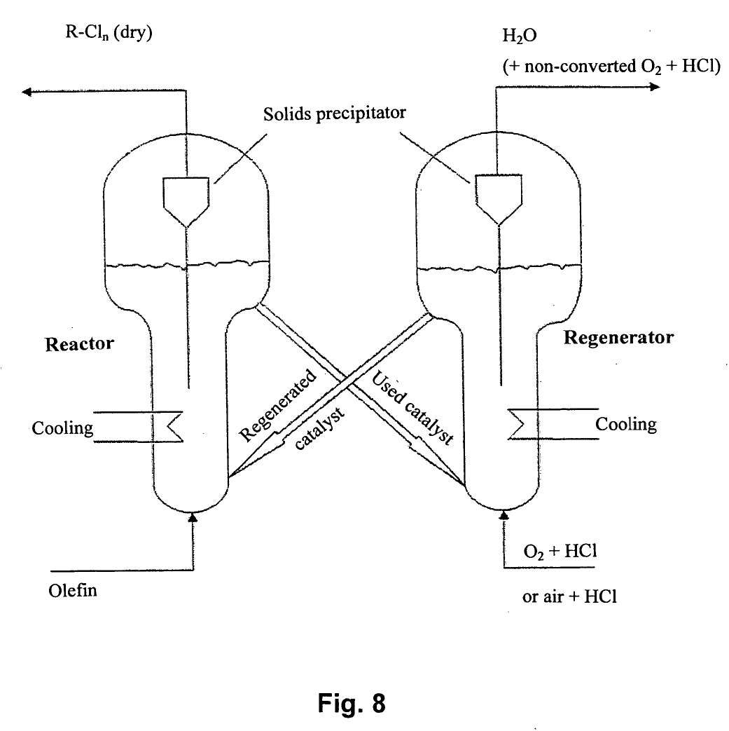

FIG. 8 shows an embodiment of the reactor to carry out the process according to the invention using separate vessels.

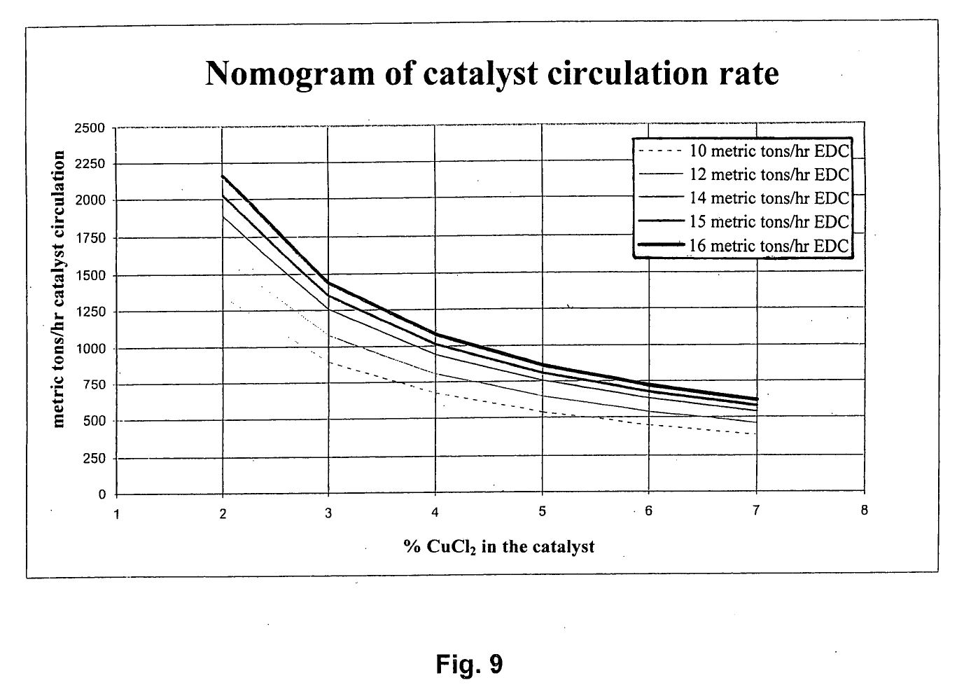

FIG. 9 shows a nomogram of the catalyst circulation rate.

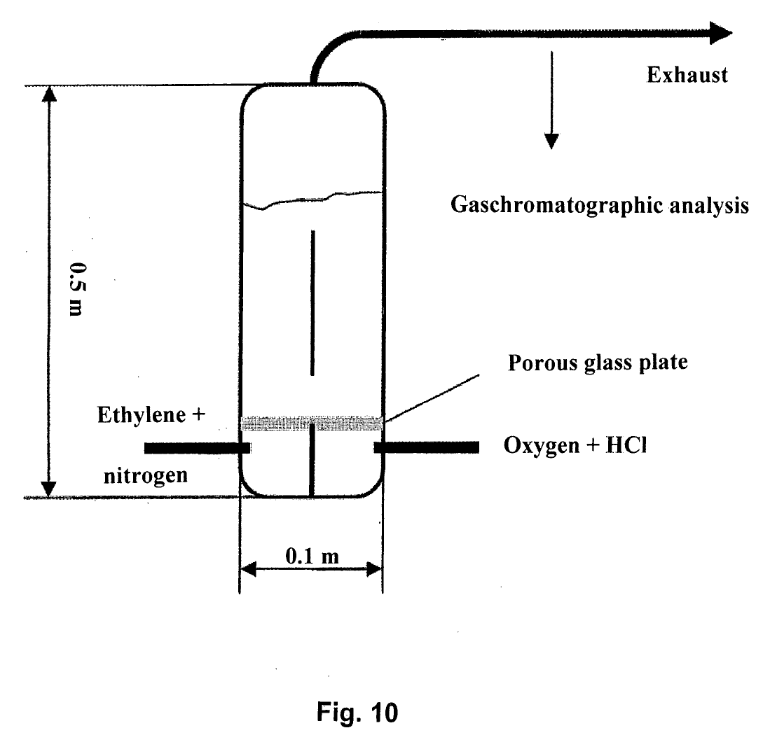

FIG. 10 shows the reactor setup used in the example.

A principal feature of the process according to the invention is the use of a reactor which has so-called reaction zones and regeneration zones. The educts, i.e. the olefins and aromatics, on the one hand, and oxygen and hydrogen chloride on the other hand are added in the respective zones and thus in a spatially separate manner. This allows better utilisation of the catalyst because under this novel fluidized bed reactor concept, olefin/aromatics and oxygen contact each other directly only to a minor extent, so that by-product generation is reduced and the oxychlorination product yield is increased. As an added bonus, the process according to the invention may be carried out at lower temperatures.

For the present invention, a “reaction zone” is a zone in the reactor which has a higher concentration rate of the catalyst in its oxidized form at the solids entry point than at the solids exit point. If, e.g., cupric chloride is used as a catalyst, this should include the components CuCl₂, CuCl and CuO at the solids entry point at ratios as specified below:

0.1 to 0.5 mol CuCl₂/kg cat; 0 to 0.1 mol CuCl/kg cat; 0 to 0.1 mol CuO/kg cat, and preferably,

0.35 mol cuCl₂/kg cat; 0.02 mol CuCl/kg cat; 0.02 mol CuO/kg cat,

At the solids exit point, the ratios are:

0.1 to 0.2 mol CuCl₂/kg cat; 0.2 to 0.3 mol CuCl/kg cat; 0 to 0.1 mol CuO/kg cat, and preferably,

0.1 mol CuCl₂/kg cat; 0.3 mol CuCl/kg cat; 0 mol CuO/kg cat.

A “regeneration zone” means a zone of the reactor which has a lower concentration rate of the catalyst in its oxidized form at the solids entry point than at the solids exit point. Accordingly, the catalyst at the solids entry point is:

0.1 to 0.2 mol CuCl₂/kg cat; 0.2 to 0.3 mol CuCl/kg cat; 0 to 0.1 mol CuO/kg Cat, and preferably,

0.1 mol CuCl₂/kg cat; 0.3 mol CuCl/kg cat; 0 mol CuO/kg cat, and at the solids exit point in general:

0.2 to 0.5 mol CuCl₂/kg cat; 0 to 0.1 mol CuCl/kg cat; 0 to 0.1 mol CuO/Kg cat, and preferably,

0.4 mol CuCl₂/kg cat; 0.05 mol CuCl/kg cat; 0.05 mol CuO/kg cat.

As already noted, the educts are fed into the reaction/regeneration zones spatially separated from each other.

For this, the olefins and aromatics are fed into the reaction zones, and oxygen/air and hydrogen chloride are fed into the regeneration zones.

In the reaction zones, the copper catalyst, which contains copper in its divalent form (Cu²⁺), is reduced to copper(I) containing forms. The reduced catalyst exits the reaction zone through circulation and passes into a regeneration zone.

Oxygen or air and hydrogen chloride are fed into the regeneration zones. There, the catalyst is returned to its original form, i.e. copper(I) salts are oxidized to copper(II) salts. The regenerated catalyst then passes out of the regeneration zone by circulation and returns to a reaction zone.

For the catalyst, any known cuprous/cupric salt catalyst which is used for oxychlorination processes may be used. Preferably, CuCl₂ is used as the catalyst for the process according to the invention.

For the process according to the invention, the catalyst circulation rate is set by controlling fluidisation in each of the reactor zones. In general, the catalyst circulation rate is 1 to 150 metric tons/hour of catalyst per metric ton/hour of product (e.g. 1,2-dichloroethane) and, preferably, some 55 metric tons/hour of catalyst per metric ton/hour of product (at a CuCl₂ content of 5 percent by mass in the oxidized catalyst).

By setting the catalyst circulation rate accordingly, it is ensured that the gaseous educts are fed into zones where the catalyst bed contains enriched reaction partners.

In order to get the catalyst bed to circulate, a driving power is required. Catalyst circulation is achieved by varying the gas velocities between areas or by forced conveyance (pump).

Gas velocity differences of 0.01 m/s to 0.1 m/s between the reaction and regeneration sides can deliver the requisite circulation rate. The geometry of the passage between the zones is a co-determinant.

The ratio between gas velocities may be between 1:1.1 and 1:1.3.

Asymmetry in fluidisation is achieved by varying the gas volumes in the zone cross-sections.

The key is the gas load in terms of area (m³/s.m²), i.e. the gas velocity (m/s). A change in the cross-section will change the gas velocity provided that the gas volume stays the same.

If oxychlorination is carried out in the circulating gas mode, the circulating gas may also be used as fluidisation gas. For the circulating gas, the gaseous non-condensable by-products (CO₂, CO), inert gases (N₂, Ar) and the non-converted educts (ethylene and oxygen) are used.

The catalyst circulating rate can be measured through the pressure distribution across the reactor width. For the process according to the invention, the catalyst circulating rate in general is:

30 to 140 metric tons/hr of catalyst circulation per metric ton/hr of product (e.g. 1,2-dichloroethane), and preferably,

50 metric tons/hr of catalyst circulation per metric ton/hr of product (e.g. 1,2-dichloroethane; corresponding to 100% of chlorine feeding from the regenerated catalyst).

The catalyst circulation achieved determines the distribution of educts across zones as follows:

If no catalyst circulates, the educts are fed evenly across the entire cross-section in line with the stoichiometry of the reaction.

If, e.g., only 50% of the chlorine required to chlorinate the ethylene dichloride used can be fed into a reaction zone through a regenerated catalyst (because the circulation rate is correspondingly low), 50% of the HCl or oxygen volume must be fed into the reaction zones. This in turn means that half the volume of ethylene needs to be fed into the regeneration zones.

If an adequate circulation rate allows all of the chlorine (100%) to be fed in the form of a regenerated catalyst, the system must be set to complete separation of the educts (i.e. 100% of the ethylene used will flow to the reaction zones, and 100% of the HCl and oxygen used will flow to the regeneration zones).

The required catalyst circulation is obtained from the quantity of chlorine to be added in accordance with the production quantities desired.

This is illustrated in the nomogram of FIG. 9. The parameters contained in it are set out in the following table.

TABLE

| | | Catalyst | | | | | | |
|---|---|---|---|---|---|---|---|---|
| | mol/kg cat | circulation | | | | | | |
| % CuCl₂ | (incl. | metric tons | | metric tons | metric tons | metric tons | metric tons | metric tons |
| (percentage | supporting | of catalyst/ | Production | per hr of | per hr of | per hr of | per hr of | per hr of |
| by mass) | material) | EDC | quantity | EDC 10 | EDC 12 | EDC 14 | EDC 15 | EDC 16 |
| 2.00 | 0.15 | 135 | Catalyst | 1349 | 1618 | 1888 | 2023 | 2158 |
| 3.00 | 0.22 | 90 | circulation | 899 | 1079 | 1259 | 1349 | 1438 |
| 4.00 | 0.30 | 67 | rate | 674 | 809 | 944 | 1011 | 1079 |
| 5.00 | 0.37 | 54 | | 539 | 647 | 755 | 809 | 863 |
| 6.00 | 0.45 | 45 | | 450 | 539 | 629 | 674 | 719 |
| 7.00 | 0.52 | 39 | | 385 | 462 | 539 | 578 | 616 |

Since, in the process according to the invention, the olefins and aromatics are no longer in direct contact with oxygen as the oxidant, the generation of oxidation products such as CO₂ and CO is inhibited. This in turn increases conversion to the desired product and reduces the volume of waste gas.

For this mode, operating temperatures are necessarily lower, at 190° C. to 210° C. Such lower reaction temperatures are made possible because the educts find a higher concentration of reaction partners (i.e. the catalyst in the respective composition) at the place of addition. With this, the reaction selectivity rises, boosting the generation of the product (e.g. 1,2-dichloroethane from ethylene). This in turn reduces the effort (energy) required for separation in the downstream cleaning lines. The quantity of high-boiling by-products to be disposed of (incinerated) declines, which in turn improves the waste gas ratio of the plant as such.

In the process according to the invention, gas feeders are arranged so that olefin and oxygen/HCl will be (almost) totally kept apart. On the other hand, an incomplete separation of educts has major advantages over the state-of-the-art processes. This means that the educt distribution can be set flexibly. Accordingly, each educt can be distributed between reaction and regeneration zones at any rate ranging from even distribution to total separation.

In an embodiment of the process according to the invention, this is achieved by providing gas distributors for oxygen and HCl also in the reaction zones.

Accordingly, gas distributors for olefin may be provided alternatively or cumulatively in the regeneration zones.

The flow direction of the catalyst bed in the reaction zone is not subject to any restrictions, i.e. it may flow either counter to or in line with the bubble ascendancy direction.

The following example is provided for the purposes of illustrating the invention.

### EXAMPLE

The reactor used was the embodiment shown in FIG. 10 to carry out the process according to the invention using internal catalyst circulation.

The height of the reactor was 0.5 metres, and its diameter was 0.1 metres.

The reactor was filled with 3.1 kg of catalyst.

Next, ethylene and oxygen/HCl were added to the reactor, with the educts separated as follows:

For the gas distributor bottom, a porous plate (“frit”) was used, separated in the middle. Ethylene and nitrogen were fed through its left half. The nitrogen serves to vary the fluidisation asymmetry, since the quantities of educt must be observed in accordance with their stoichiometry. Oxygen and HCl were fed through its right half (see FIG. 10). With this, the spatial separation is achieved. (This embodiment is also feasible at a larger scale. This design is very cheap and simple. It may be built into existing plants, i.e. it is not necessary to buy a new reactor.)

The total gas volume flow through the reactor was 0.6 m³/hr to 1 m³/hr, at gas velocities of 0.02 m/s to 0.03 m/s. Between the reaction side and regeneration side, pressure differences of a range of 1 mbar to 3 mbar were measured, at a catalyst circulating rate of 0.04 kg/s.

1. A continuous process to oxychlorinate olefins and aromatics, comprising the conversion of olefins and aromatics as component (a) with oxygen and hydrogen chloride as component (b) in the presence of a solid cuprous/cupric salt catalyst in a reactor, wherein components (a) and (b) are fed separately from each other in spatial terms into reaction zones and regeneration zones of the reactor, where the reaction zone shows a higher concentration of the catalyst in its oxidized form at the solids entry point than at the solids exit point, and the regeneration zone shows a higher concentration of the catalyst in its reduced form at the solids entry point than at its solids exit point, and where component (a) is fed into the reaction zones and component (b) is fed into the regeneration zones.

2. A process according to claim 1, wherein component (b) is additionally fed into the reaction zone.

3. A process according to claim 1 wherein, component (a) is additionally fed into the regeneration zone.

4. A process according to claim 1 wherein cupric chloride is used as the catalyst.

5. A process according to claim 4, wherein the catalyst at the solids entry point of the reaction zone is 0.1 to 0.5 mol CuCl₂/kg cat; 0 to 0.1 mol CuCl/kg cat and 0 to 0.1 mol CuO/kg cat.

6. A process according to claim 5, wherein the catalyst is 0.35 mol CuCl₂/kg cat; 0.02 mol CuCl/kg cat and 0.02 mol CuO/kg cat.

7. A process according to claim 1 wherein the catalyst at the solids exit point of the reaction zone is 0.1 to 0.2 mol CuCl2/kg cat; 0.2 to 0.3 mol CuCl/kg cat and 0 to 0.1 mol CuO/kg cat.

8. A process according to claim 7, wherein the catalyst is 0.1 mol CuCl₂/kg cat; 0.3 mol CuCl/kg cat and 0 mol CuO/kg cat.

9. A process according to claim 1 wherein the catalyst at the solids entry point of the regeneration zone is 0.1 to 0.2 mol CuCl₂/kg cat; 0.2 to 0.3 mol CuCl/kg cat; 0 to 0.1 mol CuO/kg cat.

10. A process according to claim 9, wherein the catalyst is 0.1 mol CuCl₂/kg cat; 0.3 mol CuCl/kg cat and 0 mol CuO/kg cat.

11. A process according to claim 1 wherein the catalyst at the solids exit point is 0.2 to 0.5 mol CuCl₂/kg cat; 0 to 0.1 mol CuCl/kg cat and 0 to 0.1 mol CuO/kg cat.

12. A process according to claim 11, wherein the catalyst is 0.4 mol CuCl₂/kg cat; 0.05 mol CuCl/kg cat and 0.05 mol CuO/kg cat.

13. A process according to claim 1, wherein the catalyst circulation rate is 1 to 60 metric tons/hr of catalyst per metric ton/hr of product.

14. A process according to claim 13, wherein the catalyst circulation rate is 55 metric tons/hr of catalyst per metric ton/hr of product.

15. A process according to claim 1, wherein the difference in gas velocities between the reaction zones and the regeneration zones is 0.01 m/s to 0.1 m/s.

16. A process according to claim 1, wherein the ratio of gas velocities is 1:1.1 to 1:1.3.
